(19) United States
(12) Patent Application Publication
(10) Pub. No.: US 2003/0108507 A1
(43) Pub. Date: Jun. 12, 2003

(54) Shampoo compositions with anionic surfactants, amphoteric surfactants and cationic polymers

(75) Inventors: Matthew Randall Clipson, West Chester, OH (US); Michael Paul Diederich, Mariemont, OH (US); Eric Scott Johnson, Hamilton, OH (US); Nicholas William Geary, Blue Ash, OH (US); Douglas Allan Royce, Aurora, IN (US); Robert Lee Wells, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/273,818

(22) Filed: Oct. 18, 2002

Related U.S. Application Data
(60) Provisional application No. 60/330,406, filed on Oct. 18, 2001.
(60) Provisional application No. 60/385,640, filed on Jun. 4, 2002.

(51) Int. Cl. A61K 7/075; A61K 7/08

(52) U.S. Cl. 424/70.21; 424/70.22

(57) ABSTRACT

Disclosed are a hair conditioning shampoo composition comprising from about 5.0% to about 50% of an anionic surfactant, from about 0.1% to about 15% of an amphoteric surfactant wherein said amphoteric surfactant is selected from the group consisting of alkylaminoalkanoic acids, alkyliminodialkanoic acid, alkyl aminoalkanoates, and alkyliminodialkanoates, having the formula:

RR′N(CH₂)ₙCOOX

wherein R is a straight or branched alkyl or alkenyl chain from 8 to 18 carbons, R′ is a hydrogen, —(CH₂)ₙCOOX, or —(CH₂)ₘCH₃ and mixtures thereof, wherein m is 0 to 2, n is 1 to 4, and X is selected from the group consisting of hydrogen, water-soluble cations, monovalent metals, polyvalent metal cations and mixtures thereof, (c) from about 0.01% to about 5%, by weight, of a water soluble, cationic polymer hair conditioning agent, and an aqueous carrier.

### CROSS REFERENCE TO RELATED APPLICATION

The application claims the benefit of U.S. Provisional application Serial No. 60/330,406 (Case 8753P), filed on Oct. 18, 2001 and U.S. Provisional application Serial No. 60/385,640 (Case 8753P2), filed on Jun. 4, 2002 in the names of Clipson et al.

### FIELD

This invention relates to shampoo compositions containing hair conditioning ingredients.

### BACKGROUND

Human hair becomes soiled due to its contact with the surrounding atmosphere and, to a greater extent, from sebum secreted by the head. The build-up of the sebum causes the hair to have a dirty feel and an unattractive appearance. The soiling of the hair necessitates it being shampooed with frequent regularity.

Shampooing the hair cleans by removing excess soil and sebum. However, the shampooing process has disadvantages in that the hair is left in a wet, tangled and generally unmanageable state. Shampooing can also result in the hair becoming dry or “frizzy”, and a loss of luster, due to removal of natural oils or other hair moisturizing materials. After shampooing, the hair can also suffer from a loss of “softness” perceived by the user upon drying. The hair can also suffer from increased levels of static upon drying after shampooing. This can interfere with combing and can result in “fly-away” hair. A variety of approaches have been developed to alleviate the after-shampoo problems. These range from the inclusion of hair conditioning aids in shampoos to post-shampoo application of hair conditioners, i.e., hair rinses. Hair rinses are generally liquid in nature and must be applied in a separate step following the shampooing, left on the hair for a length of time, and rinsed with fresh water. This, of course, is time consuming and is not as convenient as shampoos containing both cleaning and hair conditioning ingredients.

While a wide variety of shampoos have been disclosed which contain conditioning aids, they have not been totally satisfactory for a variety of reasons. Cationic conditioning agents are highly desirable for use in hair conditioning due to their abilities to control static, improve wet detangling, and provide a silky wet hair feel to the user. One problem which has been encountered in shampoos relates to compatibility problems between good cleaning anionic surfactants and the many conventional cationic agents which historically have been used as conditioning agents. Efforts have been made to minimize adverse interaction through the use of alternate surfactants and improved cationic conditioning agents. Cationic surfactants which provide good overall conditioning in hair rinse products, in general, tend to complex with anionic cleaning surfactants and provide poor conditioning in a shampoo context. In particular, the use of soluble cationic surfactants that form soluble ionic complexes do not deposit well on the hair. Soluble cationic surfactants that form insoluble ionic complexes deposit on the hair but do not provide good hair conditioning benefits, and tend to cause the hair to have a dirty, coated feel. The use of insoluble cationic surfactants, e.g., tricetyl methyl ammonium chloride, can provide excellent anti-static benefits but do not otherwise provide good overall conditioning. Cationic polymers have been shown to be able to deliver wet conditioning benefits from shampoos. It has also been shown that low charge density polymers create greater amounts of coacervate and better wet feel benefits. It has further been found in the art, for example in U.S. Pat. No. 5,186,928, Birtwistle, Feb. 16, 1993, that higher charge density polymers are superior as deposition aids for small particle dispersed agents.

Cationic conditioning agents commonly do not provide optimal overall conditioning benefits, particularly in the area of “softness”, especially when delivered as an ingredient in a shampoo composition. Materials which can provide increased softness are nonionic silicones. Silicones in shampoo compositions have been disclosed in a number of different publications. Such publications include U.S. Pat. No. 2,826,551, Geen, issued Mar. 11, 1958; U.S. Pat. No. 3,964,500, Drakoff, issued Jun. 22, 1976; U.S. Pat. No. 4,364,837, Pader, issued Dec. 21, 1982; and British Patent 849,433, Woolston, issued Sep. 28, 1960. While these patents disclose silicone containing compositions, they do not provide a totally satisfactory product in that it is difficult to maintain the silicone well dispersed and suspended in the product. Stable, insoluble silicone-containing hair conditioning shampoo compositions have been described in U.S. Pat. No. 4,741,855, Grote and Russell, issued May 3, 1988 and U.S. Pat. No. 4,788,066, Bolich and Williams, issued Nov. 29, 1988.

Improved conditioning shampoos are provided in U.S. Pat. No. 5,573,709 issued on Nov. 12, 1996. Japanese Patent Application, Laid Open No. 56-72095, Jun. 16, 1981, Hirota et al. (Kao Soap Corp.) also discloses shampoo containing cationic polymer and silicone conditioning agents. Still other patent publications relating to shampoos with cationic agents and silicone include EPO Application Publication 0 413 417, published Feb. 20, 1991, Hartnett et al.

Additional patent publications relating to conditioning shampoos and cleansing compositions containing anionic surfactants, amphoteric surfactants, and/or cationic polymers, silicone conditioning agents are provided in U.S. Pat. No. 4,542,125 issued on Mar. 23, 1984, U.S. Pat. No. 5,409,640 issued on Jan. 31, 1994, U.S. Pat. No. 5,756,080 issued on May 26, 1998, and WO Publication 92/06669 published on Apr. 30, 1992.

Another approach to providing hair conditioning benefits to shampoo compositions has been to use materials which are oily to the touch. These materials provide improved luster and shine to the hair. Oily materials have also been combined with cationic materials in the shampoo formulations as disclosed in Japanese Patent Application Showa 53-35902, laid open Oct. 6, 1979 (Showa 54-129135), N. Uchino (Lion Yushi Co.) and Japanese Patent Application 62 [1987]-327266, filed Dec. 25, 1987, published Jul. 4, 1989, laid open No. HEI 1[1987]-168612, Horie et al.

In spite of these attempts to provide optimal combinations of cleaning ability and hair conditioning, it remains desirable to provide further improved hair conditioning shampoo compositions. For instance, cationic polymers that deliver high amounts of coacervates for wet conditioning have not been effective in acting as deposition aids for other dispersed conditioning agents..

Other patent documents which disclose shampoo compositions and a variety of conditioning agents are EPO Patent Application Publication No. 0 413 417, published Feb. 20, 1991, U.S. Pat. No. 3,964,500, Drakoff, issued Jun. 22, 1976 and U.S. Pat. No. 5,085,857 (Reid et al.).

In spite of all these approaches and attempts to provide optimum combinations of shampoos and hair conditioners, it remains desirable to provide still improved conditioning shampoos.

### SUMMARY

The present invention is directed a hair conditioning shampoo composition comprising:

(a) from about 5.0% to about 50% of an anionic surfactant;

(b) from about 0.1% to about 15% of an amphoteric surfactant wherein said amphoteric surfactant is selected from the group consisting of alkylaminoalkanoic acids, alkyliminodialkanoic acid, alkyl aminoalkanoates, and alkyliminodialkanoates, having the formula:

RR′N(CH₂)ₙCOOX

wherein R is a straight or branched alkyl or alkenyl chain from 8 to 18 carbons, R′ is a hydrogen, —(CH₂)ₙCOOX, or —(CH₂)ₘCH₃ and mixtures thereof, wherein m is 0 to 2, n is 1 to 4, and X is selected from the group consisting of hydrogen, water-soluble cations, monovalent metals, polyvalent metal cations and mixtures thereof;

(c) from about 0.01% to about 5%, by weight, of a water soluble, cationic polymer hair conditioning agent; and

(d) an aqueous carrier.

The invention, including preferred embodiments thereof, is described in further detail in the Detailed Description of the Invention, which follows.

### DETAILED DESCRIPTION OF THE INVENTION

While the specification concludes with claims which particularly point out and distinctly claim the invention, it is believed the present invention will be better understood from the following description.

The present invention addresses the need for improved conditioning shampoos, by providing a hair conditioning shampoo composition comprising from about 5.0% to about 50% of an anionic surfactant, from about 0.1% to about 15% of an amphoteric surfactant wherein said amphoteric surfactant is selected from the group consisting of alkylaminoalkanoic acids, alkyliminodialkanoic acid, alkyl aminoalkanoates, and alkyliminodialkanoates, having the formula:

RR′N(CH₂)ₙCOOX

wherein R is a straight or branched alkyl or alkenyl chain from 8 to 18 carbons, R′ is a hydrogen, —(CH₂)ₙCOOX, or —(CH₂)ₘCH₃ and mixtures thereof, wherein m is 0 to 2, n is 1 to 4, and X is selected from the group consisting of hydrogen, water-soluble cations, monovalent metals, polyvalent metal cations and mixtures thereof, from about 0.01% to about 5%, by weight, of a water soluble, cationic polymer hair conditioning agent, and an aqueous carrier.

As discussed above, it has been previously known that higher charge density polymers are superior as deposition aids for small particle dispersed agents. It is also believed that low charge density cationic polymers, although they are less efficient as deposition aids, are in fact better than the higher charge density cationic polymers for providing wet conditioning benefits.

Without being bound by theory, it is believed that the wet conditioning benefits are a result of the formation of a complex coacervate either in the full formula or during the wash or rinse step during shampoo use. This wet coacervate deposits on hair and delivers the wet conditioning benefit. Although the coacervate formation is caused by charge attraction of the anionic micelles and cationic polymers, it has been observed that the amount of this coacervate actually increases as the charge density of the cationic polymer decreases. Thus, the lower charge density cationic polymer will yield higher levels of coacervate and therefore higher wet conditioning. It is generally believed that the amount of coacervate also depends on the type of surfactants used. For instance, using only Lauryl Sulfate yields less coacervate than mixtures of Lauryl Sulfate and Laureth Sulfate which yield less coacervate than mixtures of anionic surfactants and amphoteric surfactants. It has now however been discovered that one specific type of amphoteric surfactant when combined with anionic surfactants results in formation of much greater amounts of coacervate than any previously known surfactant combination.

There exists, still, an unmet need of products that provide hair volume and body, yet still provide adequate conditioning. Surprisingly we have discovered that this can be met a synergistic mixture of the claimed surfactants and relatively low charge density polymer. While not being bound by theory, this combination yields sufficient coacervate in the product to provide slip and excellent wet detangling—yet has good rinsing qualities, minimizing rinsing to yield hair with good volume and body.

Another benefit of coacervates is that they are able to act as delivery aids for other dispersed actives in the shampoo such as silicone, anti-dandruff actives, emollients and oils. Previously it was believed that while giving improved conditioning and lathering, coacervate systems that form high levels of coacervate are the poorest as delivery aids, ie., are poorest at helping to deposit other actives. Now it has been surprisingly found that by combining specific surfactant combinations with high charge density cationic polymers, both high levels of coacervate formation and the resulting wet conditioning benefits are achieved, in addition to maintaining the high deposition aid performance of low coacervate systems.

Consequently, it has now been found that improved overall conditioning can be found by combining specific amphoteric surfactants in combination with anionic surfactants in a shampoo with a soluble cationic organic polymer hair conditioning agent. These compositions can provide improved wet while maintaining deposition consistency. Now it has been found that the components of the present invention can provide improved overall conditioning while maintaining deposition consistency.

These and other features, aspects, and advantages of the present invention will become evident to those skilled in the art from a reading of the present disclosure with the appended claims.

The essential components and properties of the compositions of the present invention are described below. A nonexclusive description of various optional and preferred components useful in embodiments of the present invention is also described below.

The shampoo compositions of the present invention can comprise, consist of, or consist essentially of the essential elements and limitations of the invention described herein, as well as any of the additional or optional ingredients, components, or limitations described herein.

All percentages, parts and ratios are based upon the total weight of the shampoo compositions of the present invention, unless otherwise specified. All such weights as they pertain to listed ingredients are based on the active level and, therefore, do not include carriers or by-products that may be included in commercially available materials, unless otherwise specified.

Herein, “soluble” refers to any material that is sufficiently soluble in water to form a substantially clear solution to the naked eye at the concentration of use of the material in water at 25° C., unless otherwise specifically indicated. Conversely, the term “insoluble” refers to all other materials that are therefore not sufficiently soluble in water to form a substantially clear solution to the naked eye at the concentration of use at 25° C., unless otherwise specifically indicated.

Herein, “liquid” refers to any visibly (by the naked eye) flowable fluid under ambient conditions (about 1 atmosphere of pressure at about 25° C.)

All cited references are incorporated herein by reference in their entireties. Citation of any reference is not an admission regarding any determination as to its availability as prior art to the claimed invention.

Detersive Surfactant

The composition of the present invention includes a detersive surfactant. The detersive surfactant component is included to provide cleaning performance to the composition. The detersive surfactant component in turn comprises anionic detersive surfactant, zwitterionic or amphoteric detersive surfactant, or a combination thereof. Such surfactants should be physically and chemically compatible with the essential components described herein, or should not otherwise unduly impair product stability, aesthetics or performance.

Suitable anionic detersive surfactant components for use in the composition herein include those which are known for use in hair care or other personal care cleansing compositions. The concentration of the anionic surfactant component in the composition should be sufficient to provide the desired cleaning and lather performance, and generally range from about 5% to about 50%, preferably from about 8% to about 30%, more preferably from about 10% to about 25%, even more preferably from about 12% to about 22%, by weight of the composition.

Preferred anionic surfactants suitable for use in the compositions are the alkyl and alkyl ether sulfates. These materials have the respective formulae ROSO₃M and RO(C₂H₄O)ₓSO₃M, wherein R is alkyl or alkenyl of from about 8 to about 18 carbon atoms, x is an integer having a value of from 1 to 10, and M is a cation such as ammonium, alkanolamines, such as triethanolamine, monovalent metals, such as sodium and potassium, and polyvalent metal cations, such as magnesium, and calcium.

Preferably, R has from about 8 to about 18 carbon atoms, more preferably from about 10 to about 16 carbon atoms, even more preferably from about 12 to about 14 carbon atoms, in both the alkyl and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. Lauryl alcohol and straight chain alcohols derived from coconut oil or palm kernel oil are preferred. Such alcohols are reacted with between about 0 and about 10, preferably from about 2 to about 5, more preferably about 3, molar proportions of ethylene oxide, and the resulting mixture of molecular species having, for example, an average of 3 moles of ethylene oxide per mole of alcohol, is sulfated and neutralized.

Other suitable anionic detersive surfactants are the water-soluble salts of organic, sulfuric acid reaction products conforming to the formula [R¹—SO₃—M] where R¹ is a straight or branched chain, saturated, aliphatic hydrocarbon radical having from about 8 to about 24, preferably about 10 to about 18, carbon atoms; and M is a cation described hereinbefore.

Still other suitable anionic detersive surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278, which descriptions are incorporated herein by reference.

Other anionic detersive surfactants suitable for use in the compositions are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic detersive surfactants include olefin sulfonates having about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non limiting example of such an alpha-olefin sulfonate mixture is described in U.S. Pat. No. 3,332,880, which description is incorporated herein by reference.

Another class of anionic detersive surfactants suitable for use in the compositions are the beta-alkyloxy alkane sulfonates. These surfactants conform to the formula

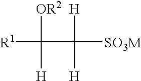

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore.

Preferred anionic detersive surfactants for use in the compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof.

Suitable amphoteric or zwitterionic detersive surfactants for use in the composition herein include those which are known for use in hair care or other personal care cleansing. Non limiting examples of suitable zwitterionic or amphoteric surfactants are described in U.S. Pat. Nos. 5,104,646 (Bolich Jr. et al.), 5,106,609 (Bolich Jr. et al.), which descriptions are incorporated herein by reference.

Alkylaminoalkanoates

Suitable amphoteric surfactant components for use in the shampoo compositions herein include alkylaminoalkanoic acids, alkyliminodialkanoic acid, alkyl aminoalkanoates, and alkyliminodialkanoates, having the formula:

RR′N(CH₂)ₙCOOX

wherein R is a straight or branched alkyl or alkenyl chain from 8 to 18 carbons, preferably R is a coconut distribution of from about 40% to 60% C₁₂, from about 10% to 30% C₁₄, and from about 2% to about 20% C₁₆, more preferably R is from C₁₂-C₁₄; R′ is a hydrogen, —(CH₂)ₙCOOX, or —(CH₂)ₘCH₃, and mixtures thereof, wherein m is 0 to 2, preferably m is 2; preferably R′ is hydrogen; n is 1 to 4, preferably n=2; and x is selected from the group consisting of hydrogen, a water-soluble cation such as ammonium, alkanolamines, such as triethanolamine, monovalent metals, such as sodium and potassium and polyvalent metal cations, such as magnesium, and calcium. Preferably, x is hydrogen.

Examples of amphoteric surfactants for use in the present shampoo compositions include cocaminopropionic acid, commercially available under the trade name Mackam 151C, cociminodipropionic acid, sodium cociminodipropionate, sodium laurylaminopropionic acid, lauraminopropionic acid, commercially available under the trade name Mackam 151L, sodium lauriminodipropionate, commercially available under the trade names Mackam 160C-30 and Mackam DP-122, laurylaminobutyric acid, sodium cocaminopropionate, sodium cocaminobutyrate, octadecylaminopropionic acid, octyliminodipropionic acid, commercially available under the tradename Mackam ODP, sodium octylaminoacetate, and potassium hexadecylaminoacetate and mixtures thereof. The preferred amphoteric surfactant is cocaminopropionic acid. The amphoteric surfactant may also contain significant amounts or some portion of unreacted alkyl amine.

Formulations with these specific amphoteric surfactants can form needle, platelet shaped crystals or unique crystals with curved shapes. These crystals undergo a transition from solid crystal to liquid crystal at near or slightly above room temperature (25° C.) and are composed of a mixture of alkyl sulfate and alkyl aminoalkanoates.

The amphoteric surfactant component will generally be present at a level from about 0.1% to about 15%, preferably from about 1% to about 7%, and more preferably from about 2% to about 5%.

Zwitterionic detersive surfactants suitable for use in the composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines are preferred. Concentration of such zwitterionic detersive surfactants preferably ranges from about 0.5% to about 20%, preferably from about 1% to about 10%, by weight of the composition.

The compositions of the present invention may further comprise additional surfactants for use in combination with the anionic detersive surfactant component described hereinbefore. Suitable optional surfactants include nonionic and cationic surfactants. Any such surfactant known in the art for use in hair or personal care products may be used, provided that the optional additional surfactant is also chemically and physically compatible with the essential components of the composition, or does not otherwise unduly impair product performance, aesthetics or stability. The concentration of the optional additional surfactants in the composition may vary with the cleansing or lather performance desired, the optional surfactant selected, the desired product concentration, the presence of other components in the composition, and other factors well known in the art.

Non limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678, 2,658,072; 2,438,091; 2,528,378, which descriptions are incorporated herein by reference.

Dispersed Particles

The composition of the present invention may include dispersed particles. In the compositions of the present invention, it is preferable to incorporate at least 0.025% by weight of the dispersed particles, more preferably at least 0.05%, still more preferably at least 0.1%, even more preferably at least 0.25%, and yet more preferably at least 0.5% by weight of the dispersed particles. In the compositions of the present invention, it is preferable to incorporate no more than about 20% by weight of the dispersed particles, more preferably no more than about 10%, still more preferably no more than 5%, even more preferably no more than 3%, and yet more preferably no more than 2% by weight of the dispersed particles.

Aqueous Carrier

The compositions of the present invention are typically in the form of pourable liquids (under ambient conditions). The compositions will therefore typically comprise an aqueous carrier, which is present at a level of from about 20% to about 95%, preferably from about 60% to about 85%, by weight of the compositions. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

Additional Components

The compositions of the present invention may further comprise one or more optional components known for use in hair care or personal care products, provided that the optional components are physically and chemically compatible with the essential components described herein, or do not otherwise unduly impair product stability, aesthetics or performance. Individual concentrations of such optional components may range from about 0.001% to about 10% by weight of the compositions.

Non-limiting examples of optional components for use in the composition include cationic polymers, conditioning agents (hydrocarbon oils, fatty esters, silicones), anti dandruff agents, suspending agents, viscosity modifiers, dyes, nonvolatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins.

Cationic Polymers

The compositions of the present invention may contain a cationic polymer. Concentrations of the cationic polymer in the composition typically range from about 0.01% to about 5%, preferably from about 0.075% to about 2.0%, more preferably from about 0.1% to about 1.0%, by weight of the composition. Preferred cationic polymers will have cationic charge densities of at least about 0.2 meq/gm, preferably at least about 0.6 meq/gm, more preferably at least about 1.5 meq/gm, but also preferably less than about 7 meq/gm, more preferably less than about 5 meq/gm, and even more preferably less than 3 meq/grm, at the pH of intended use of the composition, which pH will generally range from about pH 3 to about pH 9, preferably between about pH 4 and about pH 8. The “cationic charge density” of a polymer, as that term is used herein, refers to the ratio of the number of positive charges on the polymer to the molecular weight of the polymer. The average molecular weight of such suitable cationic polymers will generally be between about 10,000 and 10 million, preferably between about 50,000 and about 5 million, more preferably between about 100,000 and about 3 million.

Suitable cationic polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Non limiting examples of such counterions include halides (e.g., chloride, fluoride, bromide, iodide), sulfate and methylsulfate.

Non limiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which description is incorporated herein by reference.

Non limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). Preferred cationic substituted monomers are the cationic substituted dialkylaminoalkyl acrylamides, dialkylaminoalkyl methacrylamides, and combinations thereof. These preferred monomers conform the to the formula:

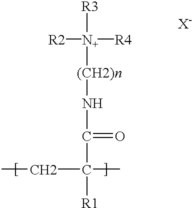

wherein R¹ is hydrogen, methyl or ethyl; each of R², R³ and R⁴ are independently hydrogen or a short chain alkyl having from about 1 to about 8 carbon atoms, preferably from about 1 to about 5 carbon atoms, more preferably from about 1 to about 2 carbon atoms; n is an integer having a value of from about 1 to about 8, preferably from about 1 to about 4; and X is a counterion. The nitrogen attached to R², R³ and R⁴ may be a protonated amine (primary, secondary or tertiary), but is preferably a quaternary ammonium wherein each of R², R³ and R⁴ are alkyl groups a non limiting example of which is polymethyacrylamidopropyl trimonium chloride, available under the trade name Polycare 133, from Rhone-Poulenc, Cranberry, N.J., U.S.A. Also preferred are copolymers of the above cationic monomer with nonionic monomers such that the charge density of the total copolymers is about 2.0 to about 4.5 meq/gram.

Other suitable cationic polymers for use in the composition include polysaccharide polymers, such as cationic cellulose derivatives and cationic starch derivatives. Suitable cationic polysaccharide polymers include those which conform to the formula:

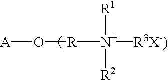

wherein A is an anhydroglucose residual group, such as a starch or cellulose anhydroglucose residual; R is an alkylene oxyalkylene, polyoxyalkylene, or hydroxyalkylene group, or combination thereof; R1, R2, and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) preferably being about 20 or less; and X is an anionic counterion as described in hereinbefore.

Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable types of cationic cellulose includes the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include cationic guar gum derivatives, such as guar hydroxypropyltrimonium chloride, specific examples of which include the Jaguar series commercially available from Rhone-Poulenc Incorporated and the N-Hance series commercially available from Aqualon Division of Hercules, Inc. Other suitable cationic polymers include quaternary nitrogen-containing cellulose ethers, some examples of which are described in U.S. Pat. No. 3,962,418, which description is incorporated herein by reference herein. Other suitable cationic polymers include copolymers of etherified cellulose, guar and starch, some examples of which are described in U.S. Pat. No. 3,958,581, which description is incorporated herein by reference. When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

As discussed above, the cationic polymer hereof is water soluble. This does not mean, however, that it must be soluble in the shampoo composition. Preferably however, the cationic polymer is either soluble in the shampoo composition, or in a complex coacervate phase in the shampoo composition formed by the cationic polymer and anionic material. Complex coacervates of the cationic polymer can be formed with anionic surfactants or with anionic polymers that can optionally be added to the compositions hereof (e.g., sodium polystyrene sulfonate).

Coacervate formation is dependent upon a variety of criteria such as molecular weight, concentration, and ratio of interacting ionic materials, ionic strength (including modification of ionic strength, for example, by addition of salts), charge density of the cationic and anionic species, pH, and temperature. Coacervate systems and the effect of these parameters have previously been studied. See, for example, J. Caelles, et al., “Anionic and Cationic Compounds in Mixed Systems”, Cosmetics & Toiletries, Vol. 106, April 1991, pp 49-54, C. J. van Oss, “Coacervation, Complex-Coacervation and Flocculation”, J. Dispersion Science and Technology, Vol. 9 (5,6), 1988-89, pp 561-573, and D. J. Burgess, “Practical Analysis of Complex Coacervate Systems”, J. of Colloid and Interface Science, Vol. 140, No. 1, November 1990, pp 227-238.

Complex coacervates are believed to readily deposit on the hair. Thus, in general, it is preferred that the cationic polymer exist in the shampoo as a coacervate phase or form a coacervate phase upon dilution. If not already a coacervate in the shampoo, the cationic polymer will preferably exist in a complex coacervate form in the shampoo upon dilution with water to a water:shampoo composition weight ratio of about 20:1, more preferably at about 10:1, even more preferably at about 5:1.

Techniques for analysis of formation of complex coacervates are known in the art. For example, microscopic analyses of the shampoo compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

In the present invention, the hair conditioning shampoo composition comprises from about 1% to 30% of an anionic surfactant, from about 0.5% to about 20% of an amphoteric surfactant, and from about 0.01% to about 5% of a cationic polymer wherein the cationic polymer and the surfactant system form a coacervate phase in the shampoo or upon dilution of the shampoo composition and the Coacervate Centrifugation Level, without the presence of carbopol-like polymers, is ≧40% as measured by the coacervate centrifugation test, preferably the Coacervate Centrifugation Level is ≧50%. The coacervate which is formed in the present invention is able to give an Active Deposition Efficiency of at least 200 PPM/% active level in the shampoo for dispersed actives having a particle size of ≦2μ as measured in a standard hair deposition test, preferably at least 300 PPM/% active level in the shampoo for dispersed actives having a particle size of ≦2μ.

A dispersed active is a benefit agent material that is insoluble in the shampoo composition and exists as particles or droplets suspended in the shampoo composition.

The Coacervate Centrifugation Level is measured using the coacervate centrifugation test. This test applies only to products that do no contain carbopol. Products with carbopol give an excessively high result on this test, but do not provide the conditioning or deposition aid benefits seen here. In this test the shampoo is diluted 1:9 with tap water. The diluted shampoo is mixed slowly for at least 2 hours and then centrifuged at 9000 Gravities force for 20 minutes. The supernate phase (top phase) is then removed and the weight of the coacervate phase (bottom phase) is measured. The percent coacervate is calculated from the equation below:

$$Coacervate Centrifugation Level = \frac{100 \times weight of coacervate phase}{( weight of diluted shampoo / 10 )}$$

The percent coacervate calculation is based on the amount of the coacervate as a function of the amount of shampoo used in the test.

The standard deposition test takes a switch of hair and shampoos the switch with 6 lather/rinse cycles (applying 0.1 grams of shampoo per gram of hair in each cycle). The switch is dried and then the amount of the specific active, such as silicone, deposited on the hair, is measured by an appropriated analytical method for the specific active being evaluated.

Exemplary complex coacervate shampoo compositions are shown in the examples. Many other cationic polymers, depending upon the other parameters of the shampoo composition, can also form coacervates, as will be understood by those skilled in the art.

It has been found that for compositions containing cationic polymer conditioning agents having cationic charge density and molecular weight within the above range can provide enhanced conditioning performance and coacervate formation.

Nonionic Polymers

Polyalkylene glycols having a molecular weight of more than about 1000 are useful herein. Useful are those having the following general formula:

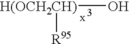

wherein R⁹⁵ is selected from the group consisting of H, methyl, and mixtures thereof. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

Conditioning Agents

Conditioning agents include any material which is used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. The conditioning agents useful in the compositions of the present invention typically comprise a water insoluble, water dispersible, non-volatile, liquid that forms emulsified, liquid particles or are solubilized by the surfactant micelles, in the anionic detersive surfactant component (described herein). Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

The concentration of the conditioning agent in the composition should be sufficient to provide the desired conditioning benefits, and as will be apparent to one of ordinary skill in the art. Such concentration can vary with the conditioning agent, the conditioning performance desired, the average size of the conditioning agent particles, the type and concentration of other components, and other like factors.

1. Silicones

The conditioning agent of the compositions of the present invention is preferably an insoluble silicone conditioning agent. The silicone conditioning agent particles may comprise volatile silicone, non-volatile silicone, or combinations thereof. Preferred are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

The concentration of the silicone conditioning agent typically ranges from about 0.01% to about 10%, by weight of the composition, preferably from about 0.1% to about 8%, more preferably from about 0.1% to about 5%, most preferably from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609, which descriptions are incorporated herein by reference. The silicone conditioning agents for use in the compositions of the present invention preferably have a viscosity, as measured at 25° C., from about 20 to about 2,000,000 centistokes (“csk”), more preferably from about 1,000 to about 1,800,000 csk, even more preferably from about 50,000 to about 1,500,000 csk, most preferably from about 100,000 to about 1,500,000 csk.

The dispersed silicone conditioning agent particles typically have a number average particle diameter ranging from about 0.01 μm to about 50 μm. For small particle application to hair, the number average particle diameters typically range from about 0.01 μm to about 4 μm, preferably from about 0.01 μm to about 2 μm, more preferably from about 0.01 μm to about 0.5 μm. Such small particle application to the hair may include the use of a deposition aide. For larger particle application to hair, the number average particle diameters typically range from about 4 μm to about 50 μm, preferably from about 6 μm to about 30 μm, more preferably from about 9 μm to about 20 μm, most preferably from about 12 μm to about 18 μm. The insoluble hair conditioning particles useful in the present invention may have a particle size range less than or equal to 35 microns, preferably less than or equal to 10 microns, even more preferably less than or equal to 2 microns.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

a. Silicone Oils

Silicone fluids include silicone oils, which are flowable silicone materials having a viscosity, as measured at 25° C., less than 1,000,000 csk, preferably from about 5 csk to about 1,000,000 csk, more preferably from about 100 csk to about 600,000 csk. Suitable silicone oils for use in the compositions of the present invention include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

Silicone oils include polyalkyl or polyaryl siloxanes which conform to the following Formula (III):

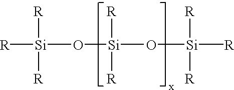

wherein R is aliphatic, preferably alkyl or alkenyl, or aryl, R can be substituted or unsubstituted, and x is an integer from 1 to about 8,000. Suitable R groups for use in the compositions of the present invention include, but are not limited to: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Preferred alkyl and alkenyl substituents are C₁ to C₅ alkyls and alkenyls, more preferably from C₁ to C₄, most preferably from C₁ to C₂. The aliphatic portions of other alkyl-, alkenyl-, or alkynyl-containing groups (such as alkoxy, alkaryl, and alkamino) can be straight or branched chains, and are preferably from C₁ to C₅, more preferably from C₁ to C₄, even more preferably from C₁ to C₃, most preferably from C₁ to C₂. As discussed above, the R substituents can also contain amino functionalities (e.g. alkamino groups), which can be primary, secondary or tertiary amines or quaternary ammonium. These include mono-, di- and tri-alkylamino and alkoxyamino groups, wherein the aliphatic portion chain length is preferably as described herein.

b. Amino and Cationic Silicones

Cationic silicone fluids suitable for use in the compositions of the present invention include, but are not limited to, those which conform to the general formula (V):

(R₁)ₐG₃₋ₐ—Si—(—OSiG₂)ₙ—(—OSiG_{b}(R₁)_{2-b)m}—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 0; b is 0 or 1, preferably 1; n is a number from 0 to 1,999, preferably from 49 to 499; m is an integer from 1 to 2,000, preferably from 1 to 10; the sum of n and m is a number from 1 to 2,000, preferably from 50 to 500; R₁ is a monovalent radical conforming to the general formula CqH_{2q}L, wherein q is an integer having a value from 2 to 8 and L is selected from the following groups:

—N(R₂)CH₂—CH₂—N(R₂)₂

—N(R₂)₂

—N(R₂)₃A⁻

—N(R₂)CH₂—CH₂—NR₂H₂A⁻

wherein R₂ is hydrogen, phenyl, benzyl, or a saturated hydrocarbon radical, preferably an alkyl radical from about C₁ to about C₂₀, and A⁻ is a halide ion.

An especially preferred cationic silicone corresponding to formula (V) is the polymer known as “trimethylsilylamodimethicone”, which is shown below in formula (VI):

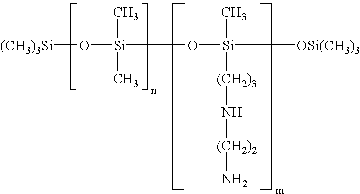

Other silicone cationic polymers which may be used in the compositions of the present invention are represented by the general formula (VII):

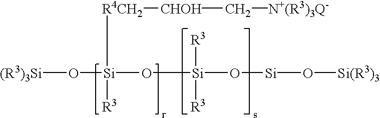

wherein R³ is a monovalent hydrocarbon radical from C₁ to C₁₈, preferably an alkyl or alkenyl radical, such as methyl; R₄ is a hydrocarbon radical, preferably a C₁ to C₁₈ alkylene radical or a C₁₀ to C₁₈ alkyleneoxy radical, more preferably a C₁ to C₈ alkyleneoxy radical; Q⁻ is a halide ion, preferably chloride; r is an average statistical value from 2 to 20, preferably from 2 to 8; s is an average statistical value from 20 to 200, preferably from 20 to 50. A preferred polymer of this class is known as UCARE SILICONE ALE 56™, available from Union Carbide.

c. Silicone Gums

Other silicone fluids suitable for use in the compositions of the present invention are the insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76, all of which are incorporated herein by reference. Specific non-limiting examples of silicone gums for use in the compositions of the present invention include polydimethylsiloxane, (polydimethylsiloxane)(methylvinylsiloxane) copolymer, poly(dimethylsiloxane)(diphenylsiloxane)(methylvinylsiloxane) copolymer and mixtures thereof.

d. High Refractive Index Silicones

Other non-volatile, insoluble silicone fluid conditioning agents that are suitable for use in the compositions of the present invention are those known as “high refractive index silicones,” having a refractive index of at least about 1.46, preferably at least about 1.48, more preferably at least about 1.52, most preferably at least about 1.55. The refractive index of the polysiloxane fluid will generally be less than about 1.70, typically less than about 1.60. In this context, polysiloxane “fluid” includes oils as well as gums.

The high refractive index polysiloxane fluid includes those represented by general Formula (III) above, as well as cyclic polysiloxanes such as those represented by Formula (VIII) below:

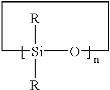

wherein R is as defined above, and n is a number from about 3 to about 7, preferably from about 3 to about 5.

The high refractive index polysiloxane fluids contain an amount of aryl-containing R substituents sufficient to increase the refractive index to the desired level, which is described herein. Additionally, R and n must be selected so that the material is non-volatile.

Aryl-containing substituents include those which contain alicyclic and heterocyclic five and six member aryl rings and those which contain fused five or six member rings. The aryl rings themselves can be substituted or unsubstituted.

Generally, the high refractive index polysiloxane fluids will have a degree of aryl-containing substituents of at least about 15%, preferably at least about 20%, more preferably at least about 25%, even more preferably at least about 35%, most preferably at least about 50%. Typically, the degree of aryl substitution will be less than about 90%, more generally less than about 85%, preferably from about 55% to about 80%.

Preferred high refractive index polysiloxane fluids have a combination of phenyl or phenyl derivative substituents (most preferably phenyl), with alkyl substituents, preferably C₁-C₄ alkyl (most preferably methyl), hydroxy, or C₁-C₄ alkylamino (especially —R¹NHR²NH2 wherein each R¹ and R² independently is a C₁-C₃ alkyl, alkenyl, and/or alkoxy).

When high refractive index silicones are used in the compositions of the present invention, they are preferably used in solution with a spreading agent, such as a silicone resin or a surfactant, to reduce the surface tension by a sufficient amount to enhance spreading and thereby enhance the glossiness (subsequent to drying) of hair treated with the compositions.

Silicone fluids suitable for use in the compositions of the present invention are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984), all of which are incorporated herein by reference.

e. Silicone Resins

Silicone resins may be included in the silicone conditioning agent of the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence.

Preferred silicone resins for use in the compositions of the present invention include, but are not limited to MQ, MT, MTQ, MDT and MDTQ resins. Methyl is a preferred silicone substituent. Especially preferred silicone resins are MQ resins, wherein the M:Q ratio is from about 0.5:1.0 to about 1.5:1.0 and the average molecular weight of the silicone resin is from about 1000 to about 10,000.

The weight ratio of the non-volatile silicone fluid, having refractive index below 1.46, to the silicone resin component, when used, is preferably from about 4:1 to about 400:1, more preferably from about 9:1 to about 200:1, most preferably from about 19:1 to about 100:1, particularly when the silicone fluid component is a polydimethylsiloxane fluid or a mixture of polydimethylsiloxane fluid and polydimethylsiloxane gum as described herein. Insofar as the silicone resin forms a part of the same phase in the compositions hereof as the silicone fluid, i.e. the conditioning active, the sum of the fluid and resin should be included in determining the level of silicone conditioning agent in the composition.

2. Organic Conditioning Oils

The conditioning component of the compositions of the present invention may also comprise from about 0.05% to about 3%, by weight of the composition, preferably from about 0.08% to about 1.5%, more preferably from about 0.1% to about 1%, of at least one organic conditioning oil as the conditioning agent, either alone or in combination with other conditioning agents, such as the silicones (described herein).

a. Hydrocarbon Oils

Suitable organic conditioning oils for use as conditioning agents in the compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from Amoco Chemical Corporation. The concentration of such hydrocarbon oils in the composition preferably range from about 0.05% to about 20%, more preferably from about 0.08% to about 1.5%, and even more preferably from about 0.1% to about 1%, by weight of the composition.

b. Polyolefins

Organic conditioning oils for use in the compositions of the present invention can also include liquid polyolefins, more preferably liquid poly-α-olefins, most preferably hydrogenated liquid poly-α-olefins. Polyolefins for use herein are prepared by polymerization of C₄ to about C₁₄ olefenic monomers, preferably from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents. Preferred hydrogenated α-olefin monomers include, but are not limited to: 1-hexene to 1-hexadecenes, 1-octene to 1-tetradecene, and mixtures thereof.

c. Fatty Esters

Other suitable organic conditioning oils for use as the conditioning agent in the compositions of the present invention include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to: isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the present invention are mono-carboxylic acid esters of the general formula R′COOR, wherein R′ and R are alkyl or alkenyl radicals, and the sum of carbon atoms in R′ and R is at least 10, preferably at least 22.

Still other fatty esters suitable for use in the compositions of the present invention are di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, and adipic acid). Specific non-limiting examples of di- and tri-alkyl and alkenyl esters of carboxylic acids include isocetyl stearyol stearate, diisopropyl adipate, and tristearyl citrate.

Other fatty esters suitable for use in the compositions of the present invention are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Still other fatty esters suitable for use in the compositions of the present invention are glycerides, including, but not limited to, mono-, di-, and tri-glycerides, preferably di- and tri-glycerides, most preferably triglycerides. For use in the compositions described herein, the glycerides are preferably the mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Other fatty esters suitable for use in the compositions of the present invention are water insoluble synthetic fatty esters. Some preferred synthetic esters conform to the general Formula (IX):

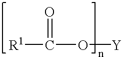

wherein R¹ is a C₇ to C₉ alkyl, alkenyl, hydroxyalkyl or hydroxyalkenyl group, preferably a saturated alkyl group, more preferably a saturated, linear, alkyl group; n is a positive integer having a value from 2 to 4, preferably 3; and Y is an alkyl, alkenyl, hydroxy or carboxy substituted alkyl or alkenyl, having from about 2 to about 20 carbon atoms, preferably from about 3 to about 14 carbon atoms. Other preferred synthetic esters conform to the general Formula (X):

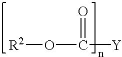

wherein R² is a C₈ to C₁₀ alkyl, alkenyl, hydroxyalkyl or hydroxyalkenyl group; preferably a saturated alkyl group, more preferably a saturated, linear, alkyl group; n and Y are as defined above in Formula (X).

Specific non-limiting examples of suitable synthetic fatty esters for use in the compositions of the present invention include: P-43 (C₈-C₁₀ triester of trimethylolpropane), MCP-684 (tetraester of 3,3 diethanol-1,5 pentadiol), MCP 121 (C₈-C₁₀ diester of adipic acid), all of which are available from Mobil Chemical Company.

3. Other Conditioning Agents

Also suitable for use in the compositions herein are the conditioning agents described by the Procter & Gamble Company in U.S. Pat. Nos. 5,674,478, and 5,750,122, both of which are incorporated herein in their entirety by reference. Also suitable for use herein are those conditioning agents described in U.S. Pat. Nos. 4,529,586 (Clairol), 4,507,280 (Clairol), 4,663,158 (Clairol), 4,197,865 (L'Oreal), 4,217,914 (L'Oreal), 4,381,919 (L'Oreal), and 4,422,853 (L'Oreal), all of which descriptions are incorporated herein by reference.

Anti-dandruff Actives

The compositions of the present invention may also contain an anti-dandruff agent. Suitable, non-limiting examples of anti-dandruff particulates include: pyridinethione salts, azoles, selenium sulfide, particulate sulfur, and mixtures thereof. Preferred are pyridinethione salts. Such anti-dandruff particulate should be physically and chemically compatible with the essential components of the composition, and should not otherwise unduly impair product stability, aesthetics or performance.

Pyridinethione Salts

Pyridinethione anti-dandruff particulates, especially 1-hydroxy-2-pyridinethione salts, are highly preferred particulate anti-dandruff agents for use in compositions of the present invention. The concentration of pyridinethione anti-dandruff particulate typically ranges from about 0.1% to about 4%, by weight of the composition, preferably from about 0.1% to about 3%, most preferably from about 0.3% to about 2%. Preferred pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), most preferably 1-hydroxy-2-pyridinethione salts in platelet particle form, wherein the particles have an average size of up to about 20μ, preferably up to about 5μ, most preferably up to about 2.5μ. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982, all of which are incorporated herein by reference. It is contemplated that when ZPT is used as the anti-dandruff particulate in the compositions herein, that the growth or re-growth of hair may be stimulated or regulated, or both, or that hair loss may be reduced or inhibited, or that hair may appear thicker or fuller.

Other Anti-microbial Actives—In addition to the anti-dandruff active selected from polyvalent metal salts of pyrithione, the present invention may further comprise one or more anti-fungal or anti-microbial actives in addition to the metal pyrithione salt actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Preferred anti-microbials include itraconazole, ketoconazole, selenium sulphide and coal tar.

Azoles

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01% to about 5%, preferably from about 0.1% to about 3%, and more preferably from about 0.3% to about 2%, by weight of the composition. Especially preferred herein is ketoconazole.

Selenium Sulfide

Selenium sulfide is a particulate anti-dandruff agent suitable for use in the anti-microbial compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), preferably less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107, all of which descriptions are incorporated herein by reference.

Sulfur

Sulfur may also be used as a particulate anti-microbial/anti-dandruff agent in the anti-microbial compositions of the present invention. Effective concentrations of the particulate sulfur are typically from about 1% to about 4%, by weight of the composition, preferably from about 2% to about 4%.

Keratolytic Agents

The present invention may further comprise one or more keratolytic agents such as Salicylic Acid.

Additional anti-microbial actives of the present invention may include extracts of melaleuca (tea tree) and charcoal. The present invention may also comprise combinations of anti-microbial actives. Such combinations may include octopirox and zinc pyrithione combinations, pine tar and sulfur combinations, salicylic acid and zinc pyrithione combinations, octopirox and climbasole combinations, and salicylic acid and octopirox combinations, and mixtures thereof.

Humectant

The compositions of the present invention may contain a humectant. The humectants herein are selected from the group consisting of polyhydric alcohols, water soluble alkoxylated nonionic polymers, and mixtures thereof. The humectants, when used herein, are preferably used at levels by weight of the composition of from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 1000 such as those with CTFA names PEG-200, PEG-400, PEG-600, PEG-1000, and mixtures thereof.

Suspending Agent

The compositions of the present invention may further comprise a suspending agent at concentrations effective for suspending water-insoluble material in dispersed form in the compositions or for modifying the viscosity of the composition. Such concentrations range from about 0.1% to about 10%, preferably from about 0.3% to about 5.0%, by weight of the compositions.

Suspending agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Commercially available viscosity modifiers highly useful herein include Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Hercules, ethylene oxide and/or propylene oxide based polymers with tradenames CARBOWAX PEGs, POLYOX WASRs, and UCON FLUIDS, all supplied by Amerchol.

Other optional suspending agents include crystalline suspending agents which can be categorized as acyl derivatives, long chain amine oxides, and mixtures thereof. These suspending agents are described in U.S. Pat. No. 4,741,855, which description is incorporated herein by reference. These preferred suspending agents include ethylene glycol esters of fatty acids preferably having from about 16 to about 22 carbon atoms. More preferred are the ethylene glycol stearates, both mono and distearate, but particularly the distearate containing less than about 7% of the mono stearate. Other suitable suspending agents include alkanol amides of fatty acids, preferably having from about 16 to about 22 carbon atoms, more preferably about 16 to 18 carbon atoms, preferred examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids in addition to the preferred materials listed above may be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C₁₆, C₁₈ and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide. Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

Though the suspending agent component may act to thicken the present compositions to some degree, the present compositions may also optionally contain other thickeners and viscosity modifiers such as an ethanolamide of a long chain fatty acid (e.g., polyethylene (3) glycol lauramide and coconut monoethanolamide), PEG 150 pentaerythrityl tetrastearate (Crothix) available from Croda and ammonium xylene sulfonate.

Other Optional Components

The compositions of the present invention may contain also vitamins and amino acids such as: water soluble vitamins such as vitamin B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

The compositions of the present invention may also contain pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names.

The compositions of the present invention may also contain antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

The compositions of the present invention may also contain chelating agents.

VII. Method of Manufacture

The shampoo compositions of the present invention can be prepared by using various formulation and mixing techniques or methods known in the art for preparing surfactant or conditioning compositions, or other similar compositions.

VIII. Method of Use

The shampoo compositions of the present invention are utilized conventionally, i.e., the hair is shampooed by applying an effective amount of the shampoo composition to the scalp, and then rinsing it out with water. Application of the shampoo to the scalp in general, encompasses massaging or working the shampoo in the hair such that all or most of the hair on the scalp is contacted. herein, “effective amount” means an amount which is effective in cleaning and conditioning the hair. Generally, from about 1 g to about 50 g, preferably from about 1 g to about 20 g, of the composition is applied for cleaning and conditioning the hair. Preferably, the shampoo is applied to hair in a wet or damp state.

This method for cleansing and conditioning the hair comprises the steps of:

a) wetting the hair with water, b) applying an effective amount of the shampoo composition to the hair, and c) rinsing the shampoo composition from the hair using water. These steps can be repeated as many times as desired to achieve the desired cleansing and conditioning benefit.

The compositions hereof can also be useful for cleaning and conditioning the skin. For such applications, the composition would be applied to the skin in a conventional manner, such as by rubbing or massaging the skin with the composition, optionally in the presence of water, and then rinsing it away with water.

### EXAMPLES

The following examples illustrate specific embodiments of the shampoo composition of the present invention, but are not intended to be limiting thereof. It will be appreciated that other modifications of the present invention within the skill of those in the hair care formulation art can be undertaken without departing from the spirit and scope of this invention. These exemplified embodiments of the shampoo compositions of the present invention provide cleansing of hair and improved hair conditioning performance.

All parts, percentages, and ratios herein are by weight unless otherwise specified. Some components may come from suppliers as dilute solutions. The levels given reflect the weight percent of the active material, unless otherwise specified. The excluded diluents and other materials are included in as “Minors”.

### Example 1-8

The following is a shampoo composition of the present invention:

| Supplier name/Description | 1 | 2 | 3 | 4 | 5 | 6 | 7 | 8 |
|---|---|---|---|---|---|---|---|---|
| Water-USP Purified & Minors | Q.S. to | Q.S. to | Q.S. to | Q.S. to | Q.S. to | Q.S. to | Q.S. to | Q.S. to |
| | 100 | 100 | 100 | 100 | 100 | 100 | 100 | 100 |
| Ammonium Laureth Sulfate | 14.0 | 11.7 | 10.0 | 14.0 | 8.7 | 10.0 | 8.75 | 12.5 |
| Puresyn 6 (1-decene homopolymer) | | 0.3 | | | | | | |
| Cocamide MEA | 0.8 | 0.8 | 0.8 | 0.8 | .8 | 0.8 | 0.8 | 0.8 |
| Citric Acid | 0.04 | 0.04 | 0.4 | 0.04 | 0.04 | 0.4 | 0.04 | 0.04 |
| Sodium Citrate Dihydrate | 0.4 | 0.4 | 0.4 | 0.4 | 0.4 | 0.4 | 0.4 | 0.4 |
| Disodium EDTA | 0.1 | 0.1 | 0.1 | 0.1 | 0.1 | 0.1 | 0.1 | 0.1 |
| Kathon | 0.0005 | 0.0005 | 0.0005 | 0.0005 | .0005 | 0.0005 | 0.0005 | 0.0005 |
| Sodium Benzoate | 0.25 | 0.25 | 0.25 | 0.25 | .25 | 0.25 | | |
| Cetyl Alcohol | 0.6 | 0.6 | 0.6 | 0.6 | | 0.6 | | |
| Lauryl Alcohol | | | | | 0.6 | | 0.6 | 0.6 |
| Ethylene Glycol Distearate | 1.5 | 1.5 | 1.5 | 1.5 | | 1.5 | | |
| Ammonium Lauryl Sulfate | 1.5 | 2.3 | 2.0 | 1.5 | 8.3 | 2.0 | 8.25 | 1.5 |
| Cocaminopropionic acid | 2.7 | 2.0 | | 2.7 | 1.0 | | 1.0 | 1.0 |
| C12/14 Dimethy (hydroxy) ammonium chloride | | | | | 0.15 | | 0.15 | |
| Sodium Lauraminopropionate | | | 4.0 | | | | | |
| Cocaminobutyric acid | | | | | | 4.0 | | |
| Polyquaternium-10 (KG30M) | | 0.5 | | | | | | |
| Polyquaternium-10 (JR30M) | 0.5 | | | 0.5 | | | | |
| Guar Hydroxypropyltrimonium | | | 0.35 | | | 0.35 | | |
| Chloride (Jaguar C-17) | | | | | | | | |
| Polyquaternium-10 (LR30M) | | | | | 0.5 | | 0.5 | 0.5 |
| Zinc Pyrithione | | | | 1.0 | | | | |
| Dow Corning 1664 300 nm/60 M emulsion | 2.0 | 2.0 | 1.5 | 2.0 | | 1.5 | | |
| Perfume | 0.5 | 0.5 | 0.5 | 0.5 | 0.5 | 0.5 | 0.5 | 0.5 |
| Sodium Chloride | 0-3 | 0-3 | 0-3 | 0-3 | 0-3 | 0-3 | 0-3 | 0-3 |
| Ammonium Xylene Sulfonate | 0-3 | 0-3 | 0-3 | 0-3 | 0-3 | 0-3 | 0-3 | 0-3 |

### Example 9-16

The following are shampoo compositions of the present invention (all percentages are based on weight unless otherwise specified):

| Supplier name/Description | 1 | 2 | 3 | 4 | 5 | 6 | 7 | 8 |
|---|---|---|---|---|---|---|---|---|
| Water-USP Purified & Minors | Q.S. to | Q.S. to | Q.S. to | Q.S. to | Q.S. to | Q.S. to | Q.S. to | Q.S. to |
| | 100 | 100 | 100 | 100 | 100 | 100 | 100 | 100 |
| Ammonium Laureth Sulfate | 10 | 10 | 10 | 10 | 10 | 14 | 12 | 12 |
| Puresyn 6 (1-decene homopolymer) | 0.4 | 0.4 | 0.4 | 0.4 | 0.25 | 0.4 | 0.4 | 0.4 |
| Trimethylolpropane Tricaprylate/Tricaprate | 0.1 | 0.1 | 0.1 | 0.1 | | 0.1 | 0.1 | 0.1 |
| Cocamide MEA | 0.8 | 0.8 | 0.8 | 0.8 | 0.8 | 0.8 | 0.8 | 0.8 |
| Citric Acid | 0.04 | 0.04 | 0.04 | 0.04 | 0.04 | 0.04 | 0.04 | 0.04 |
| Sodium Citrate Dihydrate | 0.4 | 0.4 | 0.4 | 0.4 | 0.4 | 0.4 | 0.4 | 0.4 |
| Disodium EDTA | 0.1 | 0.1 | 0.1 | 0.1 | 0.1 | 0.1 | 0.1 | 0.1 |
| Kathon | 0.0005 | 0.0005 | 0.0005 | 0.0005 | 0.0005 | 0.0005 | 0.0005 | 0.0005 |
| Sodium Benzoate | 0.25 | 0.25 | 0.25 | 0.25 | 0.25 | 0.25 | 0.25 | 0.25 |
| Disodium EDTA | 0.1274 | 0.1274 | 0.1274 | 0.1274 | 0.1274 | 0.1274 | 0.1274 | 0.1274 |
| Cetyl Alcohol | 0.9 | 0.9 | 0.9 | 0.9 | 0.6 | 0.6 | 0.6 | 0.6 |
| Ethylene Glycol Distearate | 1.5 | 1.5 | 1.5 | 1.5 | 1.5 | 1.5 | 1.5 | 1.5 |
| Ammonium Lauryl Sulfate | 4.0 | 4.0 | 4.0 | 4.0 | 2.0 | | 2.0 | 2.0 |
| Cocaminopropionic acid (4) | 2.0 | 2.0 | 2.0 | 2.0 | 4.0 | | | |
| Lauraminopropionic acid (5) | | | | | | 2.64 | | 1.26 |
| Sodium Lauriminodipropionate (6) | | | | | | | .93 | 0.14 |
| Polyquaternium-10 (KG30M) | | | | | 0.5 | 0.5 | 0.5 | 0.5 |
| Guar Hydroxypropyltrimonium Chloride (1) | 0.5 | | | | | | | |
| Guar Hydroxypropyltrimonium Chloride (2) | | 0.5 | | | | | | |
| Guar Hydroxypropyltrimonium Chloride (3) | | | 0.5 | | | | | |
| Polyquaternium-10 (LR30M) | | | | 0.5 | | | | |
| Dow Corning 1664 300 nm/60 M emulsion | | | | | 2.0 | | | |
| Dimethicone (Viscasil 330M) | 2.35 | 2.35 | 2.35 | 2.35 | | 2.35 | 2.35 | 2.35 |
| Perfume | 0.7 | 0.7 | 0.7 | 0.7 | 0.7 | 0.7 | 0.7 | 0.7 |
| Sodium Chloride | 0-3 | 0-3 | 0-3 | 0-3 | 0-3 | 0-3 | 0-3 | 0-3 |
| Ammonium Xylene Sulfonate | 0-3 | 0-3 | 0-3 | 0-3 | 0-3 | 0-3 | 0-3 | 0-3 |
| PEG 150 Pentaerythrityl Tetrastearate(Crothix) | 0.05 | | | | | | | |

(1) Guar having a molecular weight of about 400,000, and having a charge density of about 2.10 meq/g, available from Aqualon.
(2) Guar having a molecular weight of about 1,100,000, and having a charge density of about 2.10 meq/g, available from Aqualon.
(3) Guar having a molecular weight of about 400,000, and having a charge density of about 1.57 meq/g, available from Aqualon.
(4) Mackam 151C (40% active), McIntyre Group Ltd.
(5) Mackam 151L (40% active), McIntyre Group Ltd.
(6) Mackam 160C-30 (30% active), McIntyre Group Ltd.

The compositions illustrated in the sixteen examples were prepared in the following manner (all percentages are based on weight unless otherwise specified).

For each of the compositions, 36% of ammonium laureth sulfate (solution basis, 25% active) and 9.75% water was added to a jacketed mix tank and heated to about 74° C. with slow agitation to form a surfactant solution. Then, where present, Citric Acid, Sodium Citrate, Sodium Benzoate, Disodium EDTA, Cocamide MEA, Polyquaternium-10, Puresyn 6, Lauryl alcohol and Cetyl alcohol, were added to the tank and allowed to disperse. Ethylene glycol distearate (EGDS) was then added, with the exception of Example 5, to the mixing vessel, and melted. After the EGDS was well dispersed (after about 10 minutes) Kathon was added and mixed into the surfactant solution. This mixture was passed through a heat exchanger where it was cooled to about 35° C. and collected in a finishing tank. As a result of this cooling step, the ethylene glycol distearate when present is crystallized to form a crystalline network in the product. The remaining ingredients and remaining water were added to the finishing tank with ample agitation to insure a homogeneous mixture. Sodium Chloride or Ammonium Xylene Sulfonate were added as needed to adjust viscosity to the desired range.

Example 2 gives 73% coacervate formation as measured using the coacervate centrifugation test method and deposited 716 PPM silicone and 568 PPM ethylene glycol distearate as measured by the standard deposition test. It is understood that the examples and embodiments described herein are for illustrative purposes only and that various modifications or changes in light thereof will be suggested to one skilled in the art without departing from the scope of the invention.

What is claimed is:

1. A hair conditioning shampoo composition comprising:
(c) from about 5.0% to about 50% of an anionic surfactant;
(d) from about 0.1% to about 15% of an amphoteric surfactant wherein said amphoteric surfactant is selected from the group consisting of alkylaminoalkanoic acids, alkyliminodialkanoic acid, alkyl aminoalkanoates, and alkyliminodialkanoates, having the formula:
RR′N(CH₂)ₙCOOX
wherein R is a straight or branched alkyl or alkenyl chain from 8 to 18 carbons, R′ is a hydrogen, —(CH₂)ₙCOOX, or —(CH₂)ₘCH₃ and mixtures thereof, wherein m is 0 to 2, n is 1 to 4, and X is selected from the group consisting of hydrogen, water-soluble cations, monovalent metals, polyvalent metal cations and mixtures thereof;
(c) from about 0.01% to about 5%, by weight, of a water soluble, cationic polymer hair conditioning agent; and
(d) an aqueous carrier.

2. The shampoo composition of claim 1 wherein said amphoteric surfactant is selected from the group consisting of cocaminopropionic acid, cociminodipropionic acid, octyliminodipropionic acid, sodium lauriminodipropionate, laurylaminopropionic acid, laurylaminobutyric acid, sodium cocaminopropionate, sodium cocaminobutyrate, sodium cociminodipropionate, octadecylaminopropionic acid, sodium octylaminoacetate, and potassium hexadecylaminoacetate and mixtures thereof.

3. The shampoo composition according to claim 2 wherein the amphoteric surfactant may also comprise some portion of unreacted alkyl amines.

4. The shampoo composition of claim 2 wherein said amphoteric surfactant is cocaminopropionic acid.

5. The shampoo composition of claim 1 wherein R is C₁₂ to C₁₆.

6. The shampoo composition of claim 1 wherein R is from about 40% to about 60% C₁₂, from about 10% to about 30% C₁₄, and from about 2% to about 20% C₁₆.

7. The shampoo composition of claim 1 wherein n is 2.

8. The shampoo composition of claim 1 wherein R′ is hydrogen.

9. The shampoo composition of claim 1 wherein the anionic surfactant is selected from the group consisting of alkyl sulfates, alkyl ether sulfates, and mixtures thereof.

10. The shampoo composition of claim 1 wherein anionic surfactant is selected from the group consisting of ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate and mixtures thereof.

11. The shampoo composition of claim 1 wherein said anionic surfactant is present from about 8% to about 30%.

12. The shampoo composition of claim 11 wherein said anionic surfactant is present from about 12% to about 22%.

13. The shampoo composition of claim 1 wherein said amphoteric surfactant is present from about 1% to about 7%.

14. The shampoo composition of claim 13 wherein said amphoteric surfactant is present from about 2% to about 5%.

15. The shampoo composition of claim 1, wherein said water soluble, organic, cationic polymer has a cationic charge density from about 0.2 meq/gram to about 7 meq/gram.

16. The shampoo composition of claim 15, wherein said water soluble, organic, cationic polymer has a cationic charge density of from about 0.6 meq/gram to about 5 meq/gram.

17. The shampoo composition of claim 1, wherein the water soluble, organic, cationic polymer hair conditioning agent is a cationic cellulose polymer hair conditioning agent.

18. The shampoo composition of claim 1, wherein the water soluble, organic, cationic polymer hair conditioning agent has a molecular weight from about 10,000 to about 10 million.

19. The shampoo composition of claim 1, wherein the water soluble, organic, cationic polymer hair conditioning agent has a molecular weight from about 50,000 to about 5 million.

20. The shampoo composition of claim 1, wherein the water soluble, organic, cationic polymer hair conditioning agent has a molecular weight from about 100,000 to about 3 million.

21. The shampoo composition of claim 1, wherein the shampoo composition further comprises an insoluble hair conditioning agent.

22. The shampoo composition of claim 21, comprising from about 0.01% to about 10% of the insoluble hair conditioning agent.

23. The shampoo composition of claim 22, wherein the insoluble conditioning agent is a dispersed, insoluble, nonvolatile, nonionic silicone hair conditioning agent.

24. The shampoo composition of claim 1, further comprising a suspending agent.

25. The shampoo composition of claim 24, wherein the suspending agent is ethylene glycol distearate or carbomer.

26. The shampoo composition of claim 24 wherein the silicone hair conditioning agent comprises a polydimethylsiloxane fluid having a particle size of ≦35 microns.

27. The shampoo composition of claim 26 wherein said polydimethylsiloxane has a particle size of ≦2 microns.

28. The shampoo composition of claim 21, wherein the insoluble hair conditioning agent is selected from the group consisting of hydrocarbon oils, ethers, fatty esters, synthetic esters and mixtures thereof.

29. The shampoo composition of claim 28, wherein the fatty esters are selected from the group consisting of alkyl and alkenyl esters of fatty acids, alkyl and alkenyl esters of fatty alcohols, polyhydric alcohol esters, dicarboxylic acid esters, tricarboxylic acid esters, and mono-, di-, and tri-glycerides, and mixtures thereof.

30. The shampoo composition of claim 1, wherein the cationic polymer hair conditioning agent is selected from the group consisting of cationic cellulose, cationic starch, cationic guar, dialkylaminoalkyl acrylate, dialkylaminoalkyl methacrylate, monoalkylaminoalkyl acrylate, monoalkylaminoalkyl metharcylate, trialkyl methacryloxyalkyl ammonium salt, trialkyl acryloxyalkyl ammonium salt, diallyl quaternary ammonium salts, pyridinium, imidazolium, quaternized pyrrolidone, and polymethacrylamidopropyl-trimonium chloride and mixtures thereof.

31. The shampoo composition of claim 1, wherein the cationic polymer conditioning agent is a cationic polysaccharide polymer.

32. A hair conditioning shampoo composition comprising:
a) from about 8% to about 30% of an anionic surfactant;
b) from about 0.2% to about 10% of an amphoteric surfactant wherein said amphoteric surfactant is selected from the group consisting of alkylaminoalkanoic acids, alkyliminodialkanoic acid, alkyl aminoalkanoates, and alkyliminodialkanoates, having the formula:
RR′N(CH₂)ₙCOOX
wherein R is a straight or branched alkyl or alkenyl chain from 8 to 18 carbons, R′ is a hydrogen, —(CH₂)ₙCOOX, or —(CH₂)ₘCH₃ and mixtures thereof, wherein m is 0 to 2, n is 1 to 4, and x is selected from the group consisting of hydrogen, water-soluble cations, monovalent metals, and polyvalent metal cations and mixtures thereof;
c) from about 0.05% to about 3%, by weight, of a water soluble, cationic polymer hair conditioning agent;
d) from about 0.05% to about 5% of an insoluble hair conditioning agent wherein the insoluble hair conditioning agent is silicone;
e) a suspending agent; and
f) an aqueous carrier.

33. The shampoo composition of claim 1, which further comprises an anti-dandruff agent.

34. The shampoo composition of claim 33, wherein the anti-dandruff agent is selected from the group consisting of pyridinethione salts, selenium sulfide, particulate sulfur, ketoconazole and mixtures thereof.

35. A hair conditioning shampoo composition comprising:
a) from about 5% to about 50% of an anionic surfactant;
b) from about 0.1% to about 15% of an amphoteric surfactant;
c) from about 0.05% to about 5.0% of a dispersed active; and
d) from about 0.01% to about 5% of a cationic polymer
wherein the cationic polymer and said anionic surfactants and said amphoteric surfactants form a coacervate phase in the shampoo composition or upon dilution of the shampoo composition, wherein the Coacervate Centrifugation Level, without the presence of carbopol-like polymers, is ≧40% as measured by the coacervate centrifugation test; further wherein said coacervate gives an Active Deposition Efficiency of at least 200 PPM/% of an active level in said shampoo.

36. A hair conditioning shampoo composition according to claim 35 wherein the dispersed active has a particle size of ≦2μ as measured in a standard hair deposition test.

37. A hair conditioning shampoo composition according to claim 35 wherein said coacervate gives an Active Deposition Efficiency of at least 300 PPM/% of an active level in said shampoo.

38. A hair conditioning shampoo composition according to claim 35 wherein said composition further comprises silicone.

39. A hair conditioning shampoo composition according to claim 35 wherein said composition further comprises from about 0.1% to about 4% of zinc pyridinethione

40. A method for shampooing hair, the method comprising applying to hair an effective amount of the shampoo composition of claim 1 for cleaning and conditioning the hair and then rinsing the shampoo composition from the hair.

41. A method for shampooing hair, the method comprising applying to hair an effective amount of the shampoo composition of claim 21 for cleaning and conditioning the hair and then rinsing the composition from the hair.

42. A method for shampooing hair, the method comprising applying to hair an effective amount of the shampoo composition of claim 32 for cleaning and conditioning the hair and then rinsing the composition from the hair.

43. A method for shampooing hair, the method comprising applying to hair an effective amount of the shampoo composition of claim 35 for cleaning and conditioning the hair and then rinsing the composition from the hair.
